(19) United States
(12) Patent Application Publication
(10) Pub. No.: US 2022/0008619 A1
(43) Pub. Date: Jan. 13, 2022

(54) IMPLANT WITH CONTROLLED POROSITY MADE FROM A HYBRID MATERIAL DOPED WITH OSTEOINDUCTIVE NUTRIENT

(71) Applicants: UNIVERSITE CLERMONT AUVERGNE, Clermont-Ferrand (FR); CENTRE NATIONAL DE LA RECHERCHE SCIENTIFIQUE, Paris (FR); INSTITUT NATIONAL DE RECHERCHE POUR L'AGRICULTURE, L'ALIMENTATION ET L'ENVIRONNEMENT, Paris (FR)

(72) Inventors: Jonathan Claude Alexandre LAO, Veyre-Monton (FR); Cédric BOSSARD, Clermont-Ferrand (FR); Edouard Daniel Albert JALLOT, Saint-Beauzire (FR); Yohann WITTRANT, Chanonat (FR); Henri GRANEL, Royat (FR)

(21) Appl. No.: 17/293,868

(22) PCT Filed: Nov. 14, 2019
(86) PCT No.: PCT/EP2019/081383

§ 371 Date: May 13, 2021

(30) Foreign Application Priority Data
Nov. 15, 2018 (FR) 1860557

(51) Int. Cl. A61L 27/44; A61L 27/54; A61K 31/353; A61K 31/05; B29B 7/90; B29C 67/20

(52) U.S. Cl. CPC A61L 27/446; A61L 27/54; A61K 31/353; A61K 31/05; B29K 2105/0035; B29C 67/202; A61L 2300/216; A61L 2430/02; B29B 7/90

(57) ABSTRACT

The invention concerns an implant material for filling bone defects, bone regeneration and bone tissue engineering, an implant comprising this material, a method for manufacturing such an implant material.

The implant material of the invention comprises a hybrid material doped with an osteoinductive nutrient N comprising: a bioactive glass M made from SiO₂ and CaO, optionally containing P₂O₅ and/or optionally doped with strontium, and a biodegradable polymer P, this hybrid material being doped with an osteoinductive nutrient N.

The invention is applicable, in particular, in the medical field.

### TECHNICAL FIELD

The invention relates to an implant material for filling bone defects, for bone regeneration and for bone tissue engineering, an implant comprising this material and a method for manufacturing such an implant material.

### BACKGROUND

The global aging of the population and the accompanying disorders of the osteoarticular system make it necessary to develop high-performance bone tissue substitute materials. 18 billion euros in healthcare costs are spent each year in France on the diseases of the osteoarticular and dental systems, the musculoskeletal disorders are the most widespread occupational pathologies in the industrialized countries, while osteoporosis is on the rise in the elderly patients; these facts outline a major societal and economic challenge and explain the growing demand for biomaterials, implants with an increased lifespan capable of filling bone losses.

As the use of grafts is limited, and the materials of animal origin can pose biocompatibility problems or risks of infection, the research efforts are aimed at developing synthetic biomaterials capable of promoting bone regeneration.

These are called bioactive implants: the implanted material is not simply intended to passively fill in a bone loss by remaining as inert as possible, but rather it must stimulate, and actively participate in, the bone regeneration mechanism. This is particularly important in case of large bone defects, where the self-repair mechanism no longer functions.

Currently, the main bioactive materials used as bone substitutes are bioactive “ceramics”, such as calcium phosphates, and bioactive glasses, also known as “bioglasses”.

The first bioactive ceramics were developed by L. L. Hench (L. L. Hench et al., J. Biomed. Mater. Res. 1971, 2, 117-141; L. L. Hench et al., J. Biomed. Mater. Res. 1973, 7, 25-42).

The first bioactive glasses were prepared from SiO₂, P₂O₅, CaO and Na₂O. The silicon and phosphorus oxides are network formers that participate in the cohesion of the glass network. Alkalis and alkaline earths such as sodium and calcium do not have this capacity and modify the glass network by introducing chain breaks which are at the origin of the low melting temperature of these glasses associated with an increased structural disorder. Their presence results in a greater reactivity of the bioactive glasses through in particular their corrosion in an aqueous environment. This reactivity allows the formation of hydroxyapatite in a physiological environment and thus promotes the bone reconstruction.

The bioglass that has been studied the most is a soda-silica-phosphate-calcium glass called Bioglass® or Hench's Bioglass. Its basic composition is 45% SiO₂-24.5% CaO-24.5% Na₂O-6% P₂O₅, by weight compared to the total weight of the composition. The remarkable bioactive properties of this material are no longer to be demonstrated. Bioglass® remains at the moment one of the most interesting bioactive materials (inducing a specific response from cells).

Many developments have been made in the field of bioactive glasses since their discovery (M. Vallet-Regi et al., Eur. J. Inorg. Chem. 2003, 1029-1042), such as the incorporation of different atoms or the incorporation of active principles. The compositions of the bioactive glasses were optimized to promote the osteoblast proliferation and the bone tissue formation (WO 02/04606). The incorporation of silver has been proposed in particular to confer antibacterial properties to the bioactive glasses (WO 00/76486).

The application WO 2009/027594 describes a bioactive glass in which the strontium is introduced in quantities between 0.1 and 10% of the total weight of the bioactive glass.

These bioactive materials have the characteristic of being both biocompatible, capable of spontaneously binding to bone tissue, of promoting the adhesion of bone cells and, finally, of being bioresorbable, being progressively replaced by neoformed bone tissue as the bone regrowth proceeds.

However, despite these very satisfactory characteristics, the fragility of these materials limits their applications: although their rigidity is often greater than that of the bone, their lack of flexibility and toughness means that the bioactive materials cannot be implanted in sites subject to cyclical mechanical stress.

To overcome this defect, the patent application EP 3003414 describes an implant material for filling in bone defects, bone regeneration and bone tissue engineering, comprising a hybrid material made from a bioactive glass M made from SiO₂ and CaO, optionally containing P₂O₅ and/or optionally doped with strontium, and a biodegradable polymer P selected from:
the bioresorbable polysaccharides, preferably selected from dextran, hyaluronic acid, agar, chitosan, alginic acid, sodium or potassium alginate, galactomannan, carrageenan, pectin,
the bioresorbable polyesters, preferably polyvinyl alcohol or polylactic acid,
the biodegradable synthetic polymers, preferably a polyethylene glycol, or poly(caprolactone) (hereinafter abbreviated as PCL), and
the proteins, preferably gelatin or collagen.

This material consists of a matrix comprising the hybrid material, this matrix having at least 70% by number of pores having the shape of spheres or spherical polyhedron fitting into a sphere, the diameter of the spheres being between 100 and 900 μm, preferably between 200 and 800 μm, inclusive, with a difference between the diameter of the smallest or the largest sphere being at most 70%, preferably at most 50%, more preferably at most 30%, relative to the arithmetical mean diameter of all the spheres of the implant and the interconnections between the pores having their smallest dimension between 25 μm and 250 μm, inclusive, at least 70% in number of these pores having at least one interconnection with another pore.

The implants obtained from this material have mechanical properties close to bone tissue, and a specific morphology inspired by the trabecular bone, namely a highly porous structure consisting of a three-dimensional network of interconnected macropores of several hundred microns. Indeed, in the case of large bone defects, the bone cells need an extracellular “support” matrix capable of guiding and stimulating the cell adhesion, proliferation and differentiation, while being compatible with the vascularization and tissue invasion process.

Such a macroporous structure is also required for the new applications considered in bone tissue engineering: from cells taken from the patient, new bone tissue can be manufactured in the laboratory and subsequently re-implanted in the patient. In order to be carried out in an optimal way, this tissue culture must also be made from porous three-dimensional supports allowing a good cell adhesion, the differentiation into mature cells as well as the manufacture of the tissue and in particular the biomineralization.

Although excellent results in terms of bone regeneration have been obtained in vivo with implants made of the bioactive glass-biodegradable polymer hybrid material described in the patent application EP 3003414, the osteoinductive character of this material may not be sufficient to stimulate the regrowth of the bone tissue in certain patients suffering from disorders of the bone metabolism, such as the elderly patients or those suffering from osteoporosis.

Osteoinductive nutrients for stimulating the bone regrowth are known.

Examples of such osteoinductive nutrients are vitamin D (vitamin D2 (ergocalciferol), vitamin D3 (cholicalciferol)), vitamin D derivatives and precursors, vitamin K1, vitamin K2, omega-3 fatty acids, punicic acid, α-lipoic acid, anthocyanins, polyphenols, flavonols, procyanidins, tyrosol, oleuropein, naringenin, punicalagin, ellagic acid, phycocyanin, and hydrolysed collagen.

However, these osteoinductive nutrients are generally used as dietary supplements, in particular the polyphenols, the phenolic compounds, and more particularly e fisetin and hydroxytyrosol.

Fisetin is a polyphenol naturally present in many fruits and vegetables such as the strawberries, the apples and the cucumbers, and hydroxytyrosol is a phenolic compound naturally present in the olive.

Both compounds are known for their antioxidant properties and a diet rich in fisetin or hydroxytyrosol has been shown to strengthen the bones (Léotoing et al, The flavanoid fisetin promotes osteoblasts differentiation through Runx2 transcriptional activity, Mol. Nutr. Food Res. 58(6), 1239-1248, 2014, Mével, Elsa PhD in life and health sciences, Biomolecules, pharmacology, therapeutics, University of Nantes, Use of the procyanidins and the hydroxytyrosol in the nutritional prevention of the osteoarthritis, 2015).

However, these osteoinductive nutrients used as dietary supplements suffer from several disadvantages;

First, the bioavailability: the ingested nutrient must pass the digestive epithelium and only a fraction of the ingested amount reaches the bloodstream.

Secondly, the oral administration leads to a dilution of the nutrient in the entire bloodstream.

The low bioavailability and the dilution of the ingested nutrient implies large doses of nutrient to be administered orally.

Implants made of biodegradable polymers or polysaccharides into the network of which a drug, a biological molecule, a growth factor, etc., has been introduced are known.

Furthermore, implants on which a drug or other compound is adsorbed for a delivery directly to the site of interest are known.

But, adsorption does not result in a slow and prolonged release of the compound or drug. The release occurs with a “burst” phenomenon, i.e., an abrupt and short release.

Implants made of biodegradable polymers or polysaccharides into the network of which a drug, a biological molecule, a growth factor, etc., has been introduced are known.

However, these implants do not reproduce the structure of the bone: they do not comprise inorganic part or inorganic parts.

In this context, the invention aims at providing an implant material made of a biodegradable polymer-bioactive glass hybrid material releasing an osteoinductive nutrient usually used orally in a prolonged and regular manner over time.

### SUMMARY OF THE INVENTION

To this end, the invention provides an implant material for filling in bone defects, bone regeneration and bone tissue engineering comprising a bioactive glass M-biodegradable polymer P hybrid material doped with an osteoinductive nutrient N, preferably organic.

Preferably, in the implant material of the invention,
the biodegradable polymer P is selected from:
the bioresorbable polysaccharides, preferably selected from dextran, hyaluronic acid, agar, chitosan, alginic acid, sodium or potassium alginate, galactomannan, carrageenan, pectin,
the bioresorbable polyesters, preferably polyvinyl alcohol or polylactic acid, and
the biodegradable synthetic polymers, preferably a polyethylene glycol, or poly(caprolactone), and
the osteoinductive nutrient N is selected from vitamin D2 (ergocalciferol), vitamin D3 (cholicalciferol), vitamin D derivatives such as 25-hydroxy vitamin D2, 25-hydroxy vitamin D3, 24,25-hydroxy vitamin D2, 24,25-hydroxy vitamin D3, 1,25 dihydroxy vitamin D2, 1,25-dihydroxy vitamin D3, vitamin K1, vitamin K2, omega-3 fatty acids, punicic acid, α-lipoic acid, anthocyanins, flavonols, procyanidins, tyrosol, oleuropein, naringenin, punicalagin, ellagic acid and phycocyanin.

Also preferably, in the implant material of the invention, the weight ratio of biodegradable polymer P to bioactive glass M is between 20/80 and 80/20, inclusive, and the osteoinductive nutrient N is present in an amount between 0.1 and 10%, preferably between 0.1 and 5%, most preferably 1%, by weight relative to the total weight (bioactive glass M+biodegradable polymer P+nutrient N).

Still more preferably, the implant material of the invention consists of a matrix comprising the hybrid material doped with an osteoinductive nutrient N, this matrix having at least 70% by number of pores having the shape of spheres or polyhedron fitting into a sphere, the diameter of the spheres being between 100 and 900 μm, preferably between 200 and 800 μm, inclusive, with a difference between the diameter of the smallest or largest sphere being at most 70%, preferably at most 50%, more preferably at most 30%, relative to the arithmetical mean diameter of all the spheres of the implant and the interconnections between the pores having their smallest dimension between 25 μm and 250 μm, inclusive, at least 70% in number of pores having at least one interconnection with at least one other pore.

In a first preferred embodiment of the implant material of the invention, the hybrid material doped with an osteoinductive nutrient comprises 30% by weight of bioactive glass M made from SiO₂ and CaO, relative to the total weight (bioactive glass M+biodegradable polymer P+nutrient N), 69% by weight of poly(caprolactone), relative to the total weight (bioactive glass M+biodegradable polymer P+nutrient N), and 1% by weight of fisetin and/or hydroxytyrosol, relative to the total weight (bioactive glass M+biodegradable polymer P+nutrient N).

In a second preferred embodiment of the implant material of the invention, the hybrid material doped with an osteoinductive nutrient comprises 40% by weight of bioactive glass M made from SiO₂ and CaO, relative to the total weight (bioactive glass M+biodegradable polymer P+nutrient N), 59% by weight of poly(caprolactone), relative to the total weight (bioactive glass M+biodegradable polymer P+nutrient N), and 1% by weight of fisetin and/or hydroxytyrosol, relative to the total weight (bioactive glass M+biodegradable polymer P+nutrient N).

In all its embodiments, preferably, the implant material of the invention consists of a matrix comprising the hybrid material doped with an osteoinductive nutrient N, this matrix having at least 70% by number of pores having the shape of spheres or polyhedron fitting into a sphere, the diameter of the spheres being between 100 and 900 μm, preferably between 200 and 800 μm, inclusive, with a difference between the diameter of the smallest or largest sphere being at most 70%, preferably at most 50%, more preferably at most 30%, relative to the arithmetical mean diameter of all the spheres of the implant and the interconnections between the pores having their smallest dimension between 25 μm and 250 μm, inclusive, at least 70% in number of pores having at least one interconnection with at least one other pore.

The invention also provides an implant made of a hybrid material for filling in bone defects, bone regeneration and bone tissue engineering, which comprises an implant material according to the invention.

The implant material of the invention is obtainable by a method which is also an object of the invention.

This method for manufacturing an implant made of a hybrid material doped with an osteoinductive nutrient for filling in bone defects, bone regeneration and bone tissue engineering comprises the following steps:
a) selecting a bioactive glass M made from SiO₂ and CaO, optionally containing P₂O₅ and/or optionally doped with strontium,
b) selecting a biodegradable polymer P which is soluble in at least one solvent S1 and insoluble in at least one solvent S,
c) selecting microspheres of a pore-forming agent A having diameters and sizes corresponding to the desired diameters and sizes of the pores in the material constituting the implant to be manufactured, this pore-forming agent A being:
   made of a polymer insoluble in the at least one solvent S1 and soluble in the at least one solvent S,
d) selecting at least one osteoinductive nutrient N which is:
   soluble in at least one solvent S2 identical or different from the solvent S1, but miscible with the solvent S1, that degrades neither the biodegradable polymer P nor the bioactive glass M, and in which the microspheres of pore-forming agent A are not soluble, and
   insoluble in the solvent S,
e) introducing the microspheres of the pore-forming agent A into a mould having the desired shape and size for the implant, these microspheres forming a compact stack corresponding to the shape and the size of the pores to be obtained in the implant material, and representing at least 60% by volume, preferably at least 70% by volume relative to the total volume of the mixture of pore-forming agent A-biodegradable polymer P-alkoxy precursors of the bioactive glass M-nutrient N,
f) introducing the osteoinductive nutrient N, in solution in the solvent S2, into the biodegradable polymer P in solution in the solvent S1, and mixing,
g) introducing the mixture obtained in the step f) into the alkoxide precursors of the bioactive glass M,
h) introducing the mixture obtained in the step g) into the mould,
i) solidifying the mixture contained in the mould after the step h),
j) demoulding the mixture obtained in the step i),
k) removing the microspheres of pore-forming agent A by washing with the solvent S.

In this method, preferably:
the biodegradable polymer P is selected from:
   the bioresorbable polysaccharides, preferably selected from dextran, hyaluronic acid, agar, chitosan, alginic acid, sodium or potassium alginate, galactomannan, carrageenan, pectin,
   the bioresorbable polyesters, preferably polyvinyl alcohol or polylactic acid, and
   the biodegradable synthetic polymers, preferably a polyethylene glycol, or poly(caprolactone),
the material of the pore-forming agent A is selected from the biodegradable polymers insoluble in the at least one solvent S1 and in the at least one solvent S2. The material of the pore-forming agent A is soluble in the at least one solvent S. The material of the pore-forming agent A is preferably selected from C₁ to C₄ alkyl polymethacrylates, preferably methyl polymethacrylate or butyl polymethacrylate, polyurethane, polyglycolic acid, the various forms of polylactic acid, lactic-coglycolic acid copolymers, poly(caprolactone), polypropylene fumarate, paraffin and naphthalene, or acrylonitrile butadiene styrene (ABS),

the material of the pore-forming agent A being different from the biodegradable polymer P,
the osteoinductive nutrient N is selected from vitamin D2 (ergocalciferol), vitamin D3 (cholicalciferol), vitamin D derivatives such as 25-hydroxy vitamin D2, 25-hydroxy vitamin D3, 24,25-hydroxy vitamin D2, 24,25-hydroxy vitamin D3, 1,25 dihydroxy vitamin D2 1,25-dihydroxy vitamin D3, vitamin K1, vitamin K2, omega-3 fatty acids, punicic acid, α-lipoic acid, anthocyanins, flavonols, procyanidins, tyrosol, oleuropein, naringenin, punicalagin, ellagic acid and phycocyanin.

Also preferably, the weight ratio of biodegradable polymer P to bioactive glass M is between 20/80 and 80/20, inclusive, and the osteoinductive nutrient N is present in an amount between 0.1 and 10%, preferably between 0.1 and 5%, most preferably 1%, by weight relative to the total weight of the material obtained in the step k).

In a preferred embodiment of the method of the invention, the bioactive glass M is a glass made from SiO₂ and CaO, the biodegradable polymer P is the poly(caprolactone), the osteoinductive nutrient N is the fisetin and/or the hydroxytyrosol, the material of the microspheres of pore-forming agent A is the paraffin, the solvent S is the cyclohexane, and the solvent S1 is identical to the solvent S2 and is thetetrahydrofuran (THF).

### BRIEF DESCRIPTION OF FIGURES

The invention will be better understood and other characteristics and advantages thereof will become clearer upon reading the following explanatory description, which is made with reference to the appended Figures in which:

FIG. 1 represents a photograph taken with a scanning electron microscope of the implant obtained in example 1 (comparative), the matrix of which is made of an undoped poly(caprolactone)-bioactive glass hybrid material at a magnification of ×70,

FIG. 2 shows a photograph taken with a scanning electron microscope at a magnification of ×70 of a cross-section of the implant material obtained in example 2, consisting of a poly(caprolactone)-bioactive glass hybrid material doped with fisetin,

FIG. 3 shows in histogram form the results of the in vitro evaluation of the implants according to the invention obtained in examples 2 and 3 in comparison with the prior art implant obtained in comparative example (Example 1). Here, the differentiation of primary rat osteoblasts is evaluated by measuring the enzyme activity of the alkaline phosphatase (ALP) as evidence of the effect of the doping on the implants (Example 2 and 3),

FIG. 4 shows in graphical form the level of cell viability/proliferation of primary rat osteoblasts, after 7 days of culture in the presence of:
a control, without material (rate of 100%),
the material obtained in the example 1, and
the material obtained in the example 2,

FIG. 5 shows scanning electron microscopy images of primary rat osteoblasts (PRO) grown on:
Top photographs: human cortical bone slices at 2 different magnifications,
Middle photographs: BG-PCL disks (the material obtained in example 1), and
Bottom photographs: BG-PCL-Fis disks (the material obtained in example 2),

FIG. 6 shows the results of the implantation of the materials in a model of critical defects in mouse calvaria (craniotomy). These results were obtained by X-ray micro-tomography and allow to distinguish:
the bone defects left empty in the “control” animals,
the bone defects filled with BG-PCL (the material obtained in example 1), and
the bone defects filled with BG-PCL-Fis (the material obtained in example 2),

FIG. 7 represents the evolution of the quantification of the bone regeneration after implantation in critical defects in mouse calvaria. The results are expressed as % of new bone formation compared to the day of implantation (J0) for:
the control bone defects left empty in,
the bone defects filled with BG-PCL (the material obtained in example 1), and
the bone defects filled with BG-PCL-Fis (the material obtained in example 2).

### DETAILED DESCRIPTION OF THE INVENTION

In the foregoing and the following, the following terms have the following definitions:
“pore interconnection(s)”: opening(s) allowing the passage from one pore to another,
“aqueous medium” means any liquid medium containing water, or water alone,
“biodegradable”: degradable in a physiological liquid, for example a saline buffered solution (SBS),
“bioresorbable”: removable in a physiological medium containing biological cells,
“arithmetic mean diameter of all the pores”: sum of the diameters of the pores/number of pores,
“spherical pore” or “sphere”: pore or sphere whose ratio of the smallest diameter to the largest diameter is 0.9±0.1,
“polyhedron fitting into a sphere”: polyhedron that fits in a sphere having the same diameter at all points, the differences between the different diameters of the polyhedron fitting into a sphere being at most ±15% of the diameter of the sphere in which they fit,
“compact stack of microspheres of pore-forming agent A”: stack of microspheres of pore-forming agent A in which:

at least 70% by number, preferably more than 95% by number of microspheres are in contact with each other, and remain in contact with each other when the mixture of pore-forming agent A and biodegradable polymer P-bioactive glass M-nutrient N hybrid is in the mould, and when the stack of microspheres is covered and infiltrated with the bioactive glass M-biodegradable polymer P-nutrient N hybrid mixture.

Such a compact stack of microspheres of pore-forming agent A can be obtained by centrifuging the mixture of microspheres of pore-forming agent A and biodegradable polymer P-bioactive glass M-osteoinductive nutrient N hybrid, or by applying a negative (vacuum) or positive (above atmospheric pressure) pressure to the mixture of microspheres of pore-forming agent A and biodegradable polymer P-bioactive glass M-osteoinductive nutrient N hybrid introduced into the mould, before and during the gelling of this mixture,
“hybrid material”: material comprising a polymer phase and a bioactive glass phase, the bioactive glass phase consisting of chains of bioactive glass nanoparticles (in contrast to nanoparticles) which connect to form a three-dimensional network and which are intermingled with the polymer chains, at least one of the phases being circumscribed to domains of size smaller than a hundred of nm, thus conferring on the hybrid material the property of reacting as a single phase beyond the molecular scale,
“doped hybrid material”: material comprising a polymer phase, a bioactive glass phase, and an osteoinductive nutrient, the bioactive glass phase consisting of chains of bioactive glass nanoparticles that connect to form a three-dimensional network, and that are intermingled with the polymer chains, at least one of the phases being circumscribed to domains of size smaller than a hundred of nm, thus conferring on the hybrid material the property of reacting as a single phase beyond the molecular scale, the osteoinductive nutrient being integrated into the three-dimensional network of the bioactive glass nanoparticle chains and/or the chains of the polymer.

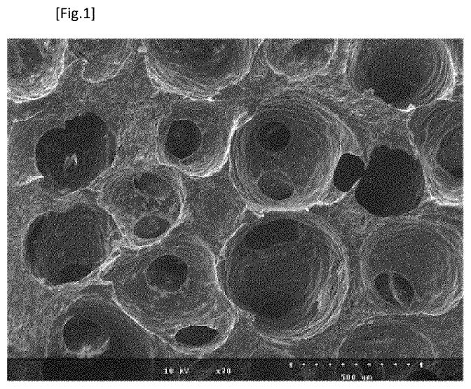

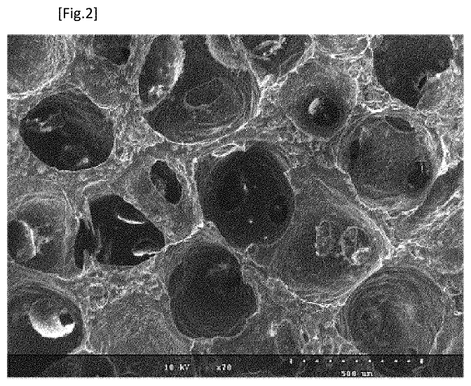

The implant material for filling in bone defects, bone regeneration and bone tissue engineering will be described in relation with FIGS. 1 and 2.

The implant material of the invention comprises a matrix of a bioactive glass M-biodegradable polymer P hybrid material doped with an osteoinductive nutrient N introduced into the network of the hybrid material.

This configuration allows to obtain a release of the osteoinductive nutrient N directly at the site of interest, in a prolonged and regular way, i.e. without any “burst” effect, because the osteoinductive nutrient N is uniformly distributed in the weight of the hybrid material and is released as the latter degrades.

The osteoinductive nutrient N is not degraded here, and thus the entire amount of this osteoinductive nutrient N reaches the site of interest.

In a preferred embodiment, the matrix of the implant material of the invention has a particular porosity which is that shown in FIG. 2.

As can be seen by comparing FIGS. 1 and 2, the implant material of the invention comprising a matrix made of a biodegradable polymer-bioactive glass hybrid material doped with an osteoinductive nutrient shown in FIG. 2 has a morphology identical to that of the implant material made of undoped biodegradable polymer-bioactive glass hybrid material of the prior art shown in FIG. 1.

It comprises a matrix of a material that comprises an organic part and an inorganic part and an osteoinductive nutrient, which is itself preferably organic.

This material is biocompatible, bioactive, bioresorbable and as seen in FIGS. 1 and 2, has a very regular morphology, in terms of pore distribution, and in terms of pore shape.

Preferably, this material has pores, in the form of spheres whose diameter, either is identical at all points, or in the form of spheres whose ratio of the smallest diameter to the largest diameter is 0.9±0.1, at most, or in the form of polyhedron fitting into such a sphere, the differences between the diameters at different points of the polyhedron fitting into this sphere being at most ±15% of the diameter of the sphere into which they fit.

The implant materials of the invention may have pore sizes in a very wide range of 100 to 900 μm, preferably 200 μm to 800 μm, inclusive, with a difference between the diameter of the smallest or largest sphere being at most 70%, preferably at most 50%, more preferably at most 30%, relative to the arithmetic mean diameter of all the spheres of the implant in association with interconnections between pores whose smallest dimension is between 25 μm and 250 μm, inclusive.

At least 70% by number of these pores have at least one interconnection with another pore.

Such a shape and distribution of pore sizes as well as such sizes of interconnections between pores are very favourable to the conduction of the cells, to the bone regrowth and to the tissue invasion as demonstrated in the patent application EP 3003414.

The matrix consists of an organic phase, an inorganic phase and an osteoinductive nutrient to increase the osteoinductive potential of the implant material of the prior art.

The inorganic phase is the bioactive glass M.

The bioactive ceramics and the bioactive glasses are well known to the person skilled in the art and are described in particular in L. L. Hench et al., J. Biomed. Mater. Res. 1971, 2, 117-141; L. L. Hench et al., J. Biomed. Mater. Res. 1973, 7, 25-42 for the bioactive ceramics and in M. Vallet-Regi et al., Eur. J. Inorg. Chem. 2003, 1029-1042 and WO 02/04606, WO 00/76486 and WO 2009/027594, in particular. In the invention, only a bioactive glass is used.

The organic part of the implant material of the invention comprises the biodegradable polymer P and the osteoinductive nutrient N. Preferably, the osteoinductive nutrient is an organic molecule.

The biodegradable polymer P is soluble in at least one solvent S1 and insoluble in at least one solvent S. These solvents may be water, an aqueous medium or an organic solvent.

Preferably, the biodegradable polymer P is selected from:
the bioresorbable polysaccharides, preferably selected from dextran, hyaluronic acid, agar, chitosan, alginic acid, sodium or potassium alginate, galactomannan, carrageenan, pectin,
the bioresorbable polyesters, preferably polyvinyl alcohol or polylactic acid, and
the biodegradable synthetic polymers, preferably a polyethylene glycol, or poly(caprolactone).

The osteoinductive nutrient N is soluble in at least one solvent S2 miscible with the solvent 51, and insoluble in the solvent S.

The solvent S2 must not degrade the properties of the final implant material: it must not lead to a separation of the organic and inorganic phases, nor must it change the homogeneity of these phases in the final material. Nor must it leads to a loss of the mechanical properties of the final implant material: the final implant material must remain manipulable, e.g. to be eventually shaped and sized for the final implant, so that the implant made of this material can be manipulated by the surgeon who is going to implant it.

Preferably, the osteoinductive nutrient N is selected from vitamin D2 (ergocalciferol), vitamin D3 (cholicalciferol), their derivatives and precursors, vitamin K1, vitamin K2, omega-3 fatty acids, punicic acid, α-lipoic acid, anthocyanins, flavonols, procyanidins, tyrosol, oleuropein, naringenin, punicalagin, ellagic acid and phycocyanin.

The matrix of the implant material of the invention consists of the bioactive glass M and the biodegradable polymer P, which form a hybrid material, i.e., forming a single phase and in which the osteoinductive nutrient N is incorporated.

In a first preferred embodiment, in the implant material of the invention, the hybrid material doped with an osteoinductive nutrient comprises 30% by weight of bioactive glass M made from SiO₂ and CaO, relative to the total weight (bioactive glass M+biodegradable polymer P+osteoinductive nutrient N), 69% by weight of poly(caprolactone), relative to the total weight (bioactive glass M+biodegradable polymer P+osteoinductive nutrient N), and 1% by weight of fisetin and/or hydroxytyrosol, relative to the total weight (bioactive glass M+biodegradable polymer P+osteoinductive nutrient N).

In a second preferred embodiment, in the implant material of the invention, the hybrid material doped with an osteoinductive nutrient comprises 40% by weight of bioactive glass M made from SiO₂ and CaO, relative to the total weight (bioactive glass M+biodegradable polymer P+osteoinductive nutrient N), 59% by weight of poly(caprolactone), relative to the total weight (bioactive glass M+biodegradable polymer P+osteoinductive nutrient N), and 1% by weight of fisetin and/or hydroxytyrosol, relative to the total weight (bioactive glass M+biodegradable polymer P+osteoinductive nutrient N).

The hybrid material used in the invention is obtained by a method which is also an object of the invention.

This method comprises forming a sol containing all the alkoxide precursors of the bioactive glass, solubilizing the biodegradable polymer P in the solvent S1, solubilizing the osteoinductive nutrient N in the solvent S2, adding the osteoinductive nutrient N solution into the solution of the biodegradable polymer P and mixing them until a uniform mixture is obtained, i.e. without phase separation, adding this biodegradable polymer P-osteoinductive nutrient N mixture into the sol containing all the alkoxide precursors of the bioactive glass and gelling the solution thus obtained by a succession of polymerization reactions (sol-gel polymerization of the inorganic phase) (alkoxide condensation). We then obtain a hybrid mixture intimately associating the mineral phase and the organic phase.

The hybrid material is thus distinguished from the composite material by an intimate entanglement between the two organic and inorganic phases, these two phases being indistinguishable (except on a molecular scale) in the case of a hybrid mixture.

In a preferred embodiment, the implant material of the invention is obtained by a method using a pore-forming agent A which consists of microspheres made of a polymer soluble in at least one solvent S in which the biodegradable polymer P and the osteoinductive nutrient N are not soluble.

In order not to degrade the mechanical and morphological properties of the implant material of the invention, the solvent S2 used to solubilize the osteoinductive nutrient N must not solubilize or degrade the pore-forming agent A. It must also not degrade the biodegradable polymer P or the bioactive glass M. Furthermore, it must be miscible with the solvent S1. Preferably, the solvent S2 is identical to the solvent S1.

Next, the method of the invention consists of stacking microspheres of pore-forming agent A made of a polymeric material, different from the biodegradable polymer P, in a mould having the shape and the size corresponding to the geometry of the bone defect to be filled or the defect where the bone regeneration is desired.

These microspheres of pore-forming agent A allow to obtain in the end pores whose size and distribution will correspond in negative to the stacking of microspheres.

In fact, the material intended to constitute the matrix will then be infiltrated into the stack of the microsphere beads of pore-forming agent A, and then solidified so that it can be demoulded without changing the shape and the size of the stack of the desired implant. The pore-forming agent A will then be removed allowing the implant material of the invention to be obtained.

As will be seen, this method does not use any high temperature heat treatment to sinter the bioactive glass M, the only temperature required being the evaporation temperature of the solvent S used.

As will become clear, the invention lies in the judicious combination of the choice of different materials and different solvents:
1) the material constituting the biodegradable polymer P,
2) the material constituting the pore-forming agent A,
3) the osteoinductive nutrient N,
4) the solvent S of the pore-forming agent A, which must not dissolve or degrade the biodegradable polymer P, the osteoinductive nutrient N and the bioactive glass M,
5) the solvents S1 and S2 which can be identical or different but which must be miscible together, not dissolve the pore-forming agent A and not degrade neither the bioactive glass M, nor the biodegradable polymer P, nor the osteoinductive nutrient N.

It is then understood that the choice of the different materials and solvents cannot be made independently of the choice of the others.

Among the preferred biodegradable polymers P that can be used are:
the bioresorbable polysaccharides, preferably selected from dextran, hyaluronic acid, agar, chitosan, alginic acid, sodium or potassium alginate, galactomannan, carrageenan, pectin;
the bioresorbable polyesters, preferably polyvinyl alcohol; or polylactic acid, and
the biodegradable synthetic polymers, preferably a polyethylene glycol, or poly(caprolactone).

While in the patent application EP3003414, the proteins are listed as a usable biodegradable polymer, in the invention the proteins are not among the usable biodegradable polymers P, due to the difficulty in solubilizing them in the presence of the osteoinductive nutrients proposed above: they are insoluble or practically insoluble in many organic solvents such as an alcohol, the tetrahydrofuran, etc., and they are poorly soluble in water.

Examples of materials for the pore-forming agent A are the biodegradable polymers insoluble in an aqueous medium and soluble in the at least one solvent S, preferably selected from C1 to C4 alkyl polymethacrylates, preferably methyl polymethacrylate or butyl polymethacrylate, polyurethane, polyglycolic acid, the various forms of polylactic acid, lactic-coglycolic acid copolymers, poly(caprolactone), polypropylene fumarate, paraffin and naphthalene, or acrylonitrile butadiene styrene (ABS).

The solvents S are in particular acetone, ethanol, chloroform, dichloromethane, hexane, cyclohexane, benzene, diethyl ether, hexafluoroisopropanol, and tetrahydrofuran (THF).

Examples of osteoinductive nutrients N are vitamin D (vitamin D2 (ergocalciferol) and vitamin D3 (cholicalciferol), its derivatives and precursors, vitamin K1, vitamin K2, omega-3 fatty acids, punicic acid, α-lipoic acid, anthocyanins, flavonols, procyanidins, tyrosol, oleuropein, naringenin, punicalagin, ellagic acid and phycocyanin.

The hydrolysed collagen is not one of the osteoinductive nutrients usable in the invention. As will be seen in the example 5 below, the implant material made of a poly(caprolactone)-bioactive glass-hydrolysed collagen hybrid material does not have sufficient mechanical strength, due to the need to employ a THF-acetic acid solvent to solubilize the PCL-hydrolysed collagen mixture.

In the invention, preferably, the biodegradable polymer P is the poly(caprolactone) (PCL), the microspheres of pore-forming agent A are made of paraffin, the solvent S is the cyclohexane, the osteoinductive nutrient N is the fisetin and/or the hydroxytyrosol, the solvent S1 is identical to the solvent S2 and is the tetrahydrofuran (THF).

In the method for manufacturing the hybrid implant material of the invention, the introduction of the microspheres into the mould can be done before the introduction of the mixture of the alkoxide precursors of the bioactive glass M, the biodegradable polymer P and the osteoinductive nutrient N.

However, it is also possible to first introduce the mixture of the alkoxide precursors of the bioactive glass M, the biodegradable polymer P and the osteoinductive nutrient N into the mould, and then pour the microspheres of pore-forming agent A into the mould.

Alternatively, a mixture of the alkoxide precursors of the bioactive glass M, the biodegradable polymer P, the osteoinductive nutrient N and the microspheres of pore-forming agent A can be made and introduced into the mould.

To obtain a material of which at least 70% by number of pores, have at least one interconnection with another pore, the amount of pore-forming agent A introduced into the biodegradable polymer P-bioactive glass M mixture must represent at least 60% by volume of the total volume of the biodegradable polymer P-bioactive glass M-osteoinductive nutrient N-pore-forming agent A mixture introduced into the mould.

The size of the interconnections is related to the size of the contact point between the spheres of pore-forming agent A in the sphere stack made. The increase in the size of the interconnections generated, at constant pore diameter, is possible by adding a step consisting of a partial fusion of the pore-forming spheres in the stack initially made, so as to increase the size of their contact point.

The microspheres of pore-forming agent A must form a compact stack, when placed in the mould with, in the invention, the sol of the alkoxide precursors of the bioactive glass M, the biodegradable polymer P and the osteoinductive nutrient N.

For this purpose, the volume of pore-forming agent A, relative to the total volume of the mixture of biodegradable polymer P-precursors of the bioactive glass M-pore-forming agent A-osteoinductive nutrient N, must be at least 60%, preferably at least 70%.

As for the ratio by weight of biopolymer P to bioactive glass M, it may be between 10/90 and 90/10. Preferably, for reasons of mechanical strength of the material obtained, it will be between 20/80 and 80/20, the best mechanical strength (easy manipulation without deformation or loss of material) being obtained with a 70/30 ratio.

The quantity of osteoinductive nutrient N can vary between 0.1% and 10%, preferably between 0.1% and 5%, by weight relative to the total weight (bioactive glass M+biodegradable polymer P+osteoinductive nutrient N).

In the case of the fisetin and the hydroxytyrosol, an amount of 1% by weight relative to the total weight (bioactive glass M+biodegradable polymer P+osteoinductive nutrient N) is sufficient.

### EXAMPLES

In order to better understand the invention, we will now describe, by way of purely illustrative and non-limitative, several implementation examples.

Example 1: (comparative) The manufacture of an implant material according to the prior art with a matrix made of undoped hybrid material in which the biodegradable polymer P is the poly(caprolactone) and the bioactive glass M is made of 75% of SiO₂ and 25% of CaO, by weight, relative to the total weight of the glass.

The first step was the stacking of the paraffin microspheres of pore-forming agent A in a mould having the desired implant geometry.

The volume of the microspheres of pore-forming agent A represented 70% of the total volume of the pore-forming agent A-biodegradable polymer P-precursor of the bioactive glass M mixture.

The pore-forming agent was in the form of spherical particles, i.e. with a diameter between 400 and 600 μm.

Their diameters can be chosen between several tens to several hundreds of microns, depending on the applications. The porosity of the implant material of the invention which will finally be obtained can be controlled on these two points; firstly the diameter of the pores which will be obtained depends directly on the diameter of the initial pore-forming particles. It is therefore sufficient to adjust the particle size of the initial paraffin microspheres in order to obtain the desired porosity very simply. Secondly, the size of the interconnections between pores depends directly on the size of the contact zone between the polymer beads in the initial stack. The size of this contact zone can be changed by fusing the initial polymer particles together, using a solvent S, or by a preliminary heat treatment. This procedure has already been described by Descamps et al., “Manufacture of macroporous beta-tricalcium phosphate bioceramics”. Journal of the European Ceramic Society 2008, 28, (1), 149-157 and “Synthesis of macroporous beta-tricalcium phosphate with controlled porous architectural”. Ceramics International 2008, 34, (5), 1131-1137.

In the second step, the poly(caprolactone) was brought into solution in tetrahydrofuran (THF).

In a third step, the poly(caprolactone) solution was poured into the sol containing the alkoxide precursors of the bioactive glass.

The alkoxide precursors of the bioactive glass were as follows.
Tetraethylorthosilicate TEOS
Calcium ethoxide Ca(OEt)₂
2M HCl.

They were in such quantities that the composition of the bioactive glass was 75% SiO₂ and 25% CaO, by weight, relative to the total weight of the bioactive glass obtained at the end.

In a fourth step, the above hybrid mixture was poured into the mould containing the paraffin microsphere stack.

A centrifugation or a pressure infiltration or a vacuum infiltration can be used to help the hybrid mixture fill the interstices between the paraffin microspheres.

The hybrid material was obtained by a sol-gel method.

In this method, a sol containing all the alkoxide precursors of the bioactive glass and the biodegradable polymer in solution is brought to gel by a succession of polymerization reactions.

This gelation is carried out at a temperature between 0° C. and 60° C., inclusive, in order not to degrade the obtained hybrid material matrix.

Once solidified, the implant material containing the paraffin microspheres is demoulded, and the paraffin microspheres of pore-forming agent are removed by washing with cyclohexane.

After several washing steps, the initial imprint of the paraffin microspheres is completely removed and the final material is obtained, in the form of a macroporous bioactive glass/poly(caprolactone) hybrid block.

An implant material was obtained made of 70% by weight of poly(caprolactone) and of 30% by weight of bioactive glass and having at least 70%, by number of pores having at least one interconnection with another pore.

The obtained structure can be washed without any damage in ethanol baths, in order to remove possible undesirable residues, such as chlorides, THF, etc.

Example 2: (according to the invention) Manufacture of an implant material with a matrix made of hybrid material doped with osteoinductive nutrient in which the biodegradable polymer P is the poly(caprolactone), the bioactive glass M is made of 75% of SiO₂ and 25% of CaO, by weight, relative to the total weight of the glass and the osteoinductive nutrient N is the fisetin.

The first step was the stacking of the paraffin microspheres of pore-forming agent A in a mould having the desired geometry for the implant.

The volume of the microspheres of pore-forming agent A was 70% of the total volume of the mixture of pore-forming agent A-biodegradable polymer P-osteoinductive nutrient N-precursors of the bioactive glass M, as in the example 1.

The pore-forming agent was in the form of spherical particles, i.e. with a diameter between 400 and 600 μm, as in the example 1.

In the second step, the poly(caprolactone) was brought in solution in tetrahydrofuran (THF), as in the example 1.

Separately, the fisetin was solubilized in the resulting poly(caprolactone) solution. This is possible because the fisetin is soluble in the THF. The mixture was stirred until a homogeneous solution was obtained, i.e. without phase separation.

The amount of fisetin introduced was such that its weight in the final implant material was 1% of the weight (bioactive glass plus poly(caprolactone) plus fisetin).

The resulting solution has a red tint due to the presence of the fisetin.

In a third step, the poly(caprolactone)—fisetin solution was poured into the sol containing the alkoxide precursors of the bioactive glass.

The alkoxide precursors of the bioactive glass were the same as those used in the example 1 and in the same amounts as in the example 1.

In a fourth step, the above hybrid mixture, was poured into the mould containing the paraffin microsphere stack.

A centrifugation or a pressure infiltration or a vacuum infiltration can be used to help the hybrid mixture fill the interstices between the paraffin microspheres.

A gelling is conducted at a temperature between 0° C. and 60° C., inclusive, so as not to degrade the resulting hybrid material matrix.

Once solidified, the implant material containing the paraffin microspheres is demoulded, and the paraffin microspheres of pore-forming agent are removed by washing with cyclohexane.

After several washing steps, the initial imprint of the paraffin microspheres is completely removed and the final material is obtained, in the form of a macroporous bioactive glass/poly(caprolactone)/fisetin hybrid block.

A reddish coloured implant material was obtained, made of 69% by weight of poly(caprolactone), 30% by weight of bioactive glass and 1% by weight of fisetin, and having 70%, by number of pores having at least one interconnection with another pore.

The obtained structure can be washed without any damage in ethanol baths, in order to remove possible undesirable residues, such as chlorides, THF, etc.

When washed with cyclohexane to remove the paraffin microspheres and with the ethanol, the solvents remain colourless, indicating a negligible or no release of the fisetin.

Example 3: (according to the invention) Manufacture of an implant material with a matrix made of hybrid material doped with osteoinductive nutrient in which the biodegradable polymer P is the poly(caprolactone), the bioactive glass M is made of 75% of SiO₂ and 25% of CaO, by weight, relative to the total weight of the glass and the osteoinductive nutrient N is the hydroxytyrosol.

The method is identical to the example 2 but replacing the fisetin with the hydroxytyrosol.

The solution obtained during the solubilization of the hydroxytyrosol in the poly(caprolactone) solution, as well as the implant material obtained at the end has a yellow tint due to the presence of the hydroxytyrosol.

Also in this example, upon washing with the cyclohexane to remove the paraffin microspheres and with the ethanol, the solvents remain colourless, indicating a negligible or no release of the hydroxytyrosol.

Example 4: (comparative) Manufacture of an implant material with a matrix made of a hybrid material in which the biodegradable polymer P is the hydrolysed collagen and the bioactive glass M is made of 75% of SiO₂ and 25% of CaO, by weight, relative to the total weight of the glass.

The hydrolysed collagen does not swell in contact with the physiological fluids.

In addition, like the gelatin and the collagen, the hydrolysed collagen has amino acid sequences that serve as receptors for the integrins and thus promote the cell adhesion.

The hydrolysed collagen is produced from the collagen. The collagen is a protein composed of three polypeptide chains, linked together by hydrogen bonds and covalent bonds. It is usually extracted from the pig or the beef skin. The partial hydrolysis of the collagen breaks the intermolecular bonds and leads to the dissociation of the strands; the gelatin is then obtained. The advanced hydrolysis of the gelatin then leads to the breaking of peptide bonds, and the strands initially made up of more than a thousand amino acids are broken down into peptides of about twenty amino acids; the hydrolysed collagen is thus obtained.

The hydrolysed collagen is dissolved in water and then mixed with the bioglass sol. The resulting hybrid sol contains white particles that give it a white colour and an opaque appearance. After homogenization by stirring and ultrasound, the hybrid sol is poured onto a stack of paraffin beads and the whole is then centrifuged at 3900 rpm for 3 min. This centrifugation does not allow the infiltration of the hydrolysed collagen-bioglass hybrid sol. Indeed, a white deposit is observed on the stack of beads, on top of which a clear solution floats.

In view of this impossibility of obtaining a homogeneous mixture of hydrolysed collagen-bioglass, studies were carried out and showed that the hydrolysed collagen and the bioglass are both soluble in acetic acid (weak organic acid)-water mixtures containing 90% of acetic acid. The chromatography-mass spectrometry analyses show that the acetic acid does not degrade the hydrolysed collagen.

The hydrolysed collagen is then dissolved at a concentration of 0.30 g/mL in an acetic acid-water mixture containing 90% by volume of acetic acid.

Then the bioglass sol is added so as to reach a weight composition of 70% polymer 30% bioglass.

The resulting hybrid solution is clear. It is then poured onto the stack of beads and centrifuged.

A gelling is carried out at a temperature between 0° C. and 60° C., inclusive, in order not to degrade the hybrid material matrix obtained.

Once solidified, the implant material containing the paraffin microspheres is demoulded, and the paraffin microspheres of pore-forming agent are removed by washing with cyclohexane.

The manufactured implant material has poor mechanical properties and dissolves rapidly in water. It is indeed necessary to handle it gently, otherwise it tends to break.

As a result, the doping of the bioglass shown here with the hydrolysed collagen is not suitable for the bone filling.

Example 5: (comparative): Manufacture of an implant material with a matrix made of a hybrid material doped with osteoinductive nutrient in which the biodegradable polymer P is the poly(caprolactone), the bioactive glass M is made of 75% of SiO₂ and 25% of CaO, by weight, relative to the total weight of the glass and the osteoinductive nutrient N is the hydrolysed collagen.

As already mentioned in the example 4, the hydrolysed collagen stimulates the activity of the osteoblasts, improves the absorption of the calcium, and has anti-inflammatory and anti-oxidant properties.

Although the synthesis of implant material of hydrolysed collagen-bioglass hybrid has been abandoned, it appeared interesting to incorporate this protein in a small proportion in the organic framework of the PCL-bioglass hybrid, an organic doping with hydrolysed collagen could improve the osteoinductive potential of the hybrid. The hydrolysed collagen is substituted for the PCL. A weight composition of 60% PCL, 10% hydrolysed collagen and 30% SiO₂—CaO bioglass is targeted, which is noted as PCL-colH.

The previous studies have revealed the acetic acid as a common solvent for the PCL and the hydrolysed collagen (see the example 4). The acetic acid allows the dissolution of the organic constituents homogeneously and the realization of the hybrid sol, but after dissolution of the paraffin beads (pore-forming) the implant materials disintegrate.

Implant materials are then manufactured using mixtures of 80% acetic acid-20% THF as a common solvent for the poly(caprolactone) and the hydrolysed collagen. They do not disintegrate and have well-defined pores and interconnections with diameters identical to those of the undoped PCL-bioglass hybrid scaffolds.

However, the cutting of the resulting implant materials is not clean and is accompanied by a slight tearing of the walls.

The release of the hydrolysed collagen is studied through a semi-dynamic interaction in the SBF (Simulated Body Fluid) at a rate of 1 mL per mg of material. The SBF is renewed every 2 days and the interacted SBF is analysed with a BioAssay kit to determine the concentration of hydrolysed collagen. The study has 6 consecutive interactions of 2 days. The analyses reveal a 40% release of the hydrolysed collagen into the SBF after the first interaction, and then the concentration of hydrolysed collagen in the SBF is below the detection limit for the subsequent interactions. These results demonstrate a rapid release of the hydrolysed collagen. The rapid release was expected since the hydrolysed collagen is water soluble and is simply intricately linked to the PCL chains (no covalent bonding). Thus, we can conclude that the potential action of this organic doping will be on the short term.

The osteoinductive potential of the PCL-colH implant material is evaluated in vitro and compared to that of the undoped hybrid (PCL-BV) and of the bovine trabecular bone (OTB). The implant materials are seeded with primary rat osteoblasts and the ALP (alkaline phosphatase) activity is determined after 7 and 14 days of cell culture. The ALP activity is significantly higher with the PCL-colH after 7 days and then is similar for the PCL-colH and the PCL-BV after 14 days. The hydrolysed collagen thus stimulates the mineralization process, but this effect is limited to short times as expected. Knowing that the SBF is renewed every 2 to 3 days in this experiment, it is possible that after 7 days of cell culture (i.e. 1 pre-incubation and 3 interactions) there is no hydrolysed collagen left in the hybrid, which does not release any more in the medium, which would explain an osteoinduction limited to early times. Thus, these cellular results are consistent with the previous observations on the rapid release of the hydrolysed collagen.

The PCL-colH implant materials are finally implanted into the calvaria of mice. After 3 months of implantation, the bone has not regenerated in the PCL-colH implant materials, whereas the bone regrowth is well advanced in the undoped implant materials. This observation correlates with an activity of the low osteoblast in the implantation area of the PCL-col H illustrated by the absence of ALP.

In addition, a significant inflammation is observed with the PCL-col H.

Therefore, the PCL-colH material does not seem to be suitable for the bone filling, in contrast to the PCL-BV hybrid, even when undoped, as shown by the in vivo performance.

Example 6: Evaluation of the mechanical properties of the implant materials obtained in the examples 1 to 3.

The manipulation of the implant materials obtained in the examples 1 to 3 shows that the doped implant materials obtained in the examples 2 and 3 have mechanical properties identical to those of the implant material of the prior art obtained in the example 1: they have good mechanical strength, they are both slightly elastic and sufficiently rigid to allow their manipulation. Their cutting with a scalpel is simple and clean. From the macroscopic point of view, it is not possible to differentiate the doped materials from the undoped material, except by the colour.

The scanning electron microscopy does not show any difference in morphology and surface finish.

As shown in FIGS. 1 and 2, the undoped implant material has the same structure as the doped implant materials, with well-defined and highly interconnected pores, a micrometric roughness on the surface of the walls, and a micrometric porosity inside the walls.

Example 7: In vitro evaluation of the implants obtained in examples 1 to 3.

The osteoinductive potentials of the hybrid implant materials doped with fisetin (PCL-fis) (Example 2) or with hydroxytyrosol (PCL-hyd) (Example 3) are evaluated in vitro and compared with that of the undoped hybrid (PCL-BV) (Example 1).

The implant materials are seeded with primary rat osteoblasts and the ALP activity is determined after 7 days and 14 days of cell culture. The ALP activity is normalized with the relative amount of GAPDH present in each sample. The culture is performed under orbital stirring (10 rpm).

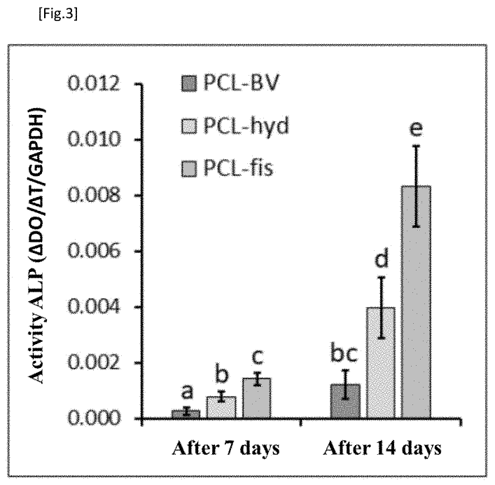

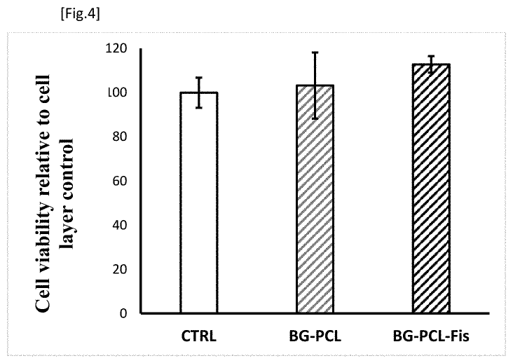

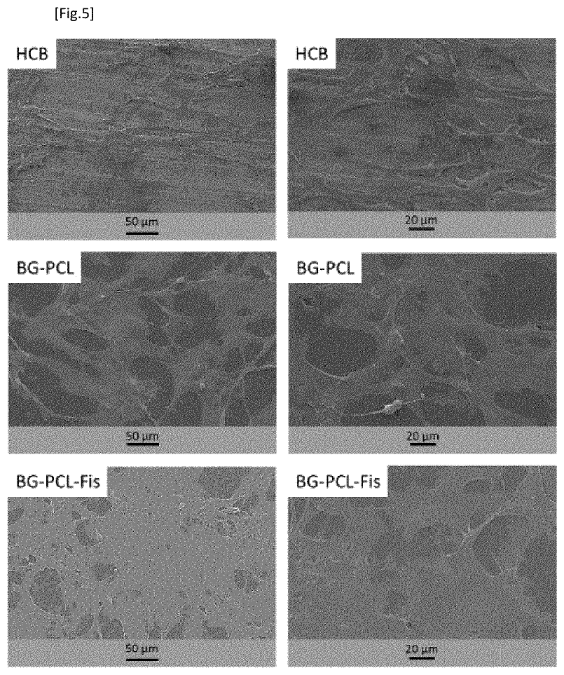

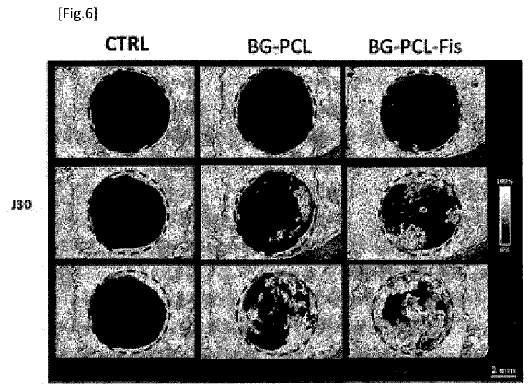

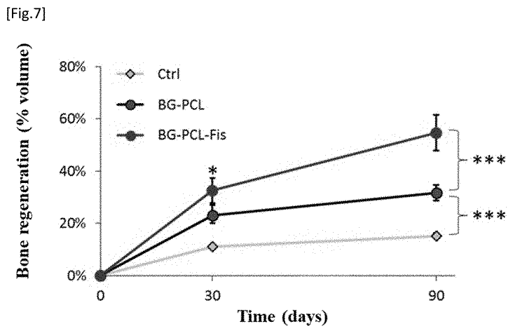

As shown in FIG. 3, which shows the quantitative results on the cell differentiation in contact with these materials, in the form of histograms (ANOVA statistical treatment followed by Tukey for a 95% confidence interval), significant differences are observed between the three materials at both times. The ALP activity is more important with PCL-fis, then PCL-hyd and finally PCL-BV, demonstrating an osteoinductive effect of these organic dopants.

The osteoinductive effect of PCL-fis and PCL-hyd is observed up to 14 days of cell culture, which is equivalent to 1 pre-incubation and 6 interactions. It seems that the release of these dopants is long lasting. A change in colour of the culture mediums after interaction with PCL-fis is observed, as they take on the colour of the dopant.

This colour change diminishes with each renewal of the medium but is still noticeable, indicating a release spread out in time. The gradual release of the fisetin and of the hydroxytyrosol can be related to a low solubility of the phenolic compounds in aqueous mediums and of the hybrid made from PCL.

In conclusion, fisetin and t hydroxytyrosol improve the osteoinductive potential of the hybrid. Also from an economic point of view, the use of these antioxidants as constituents of a biomaterial represents a much more attractive strategy compared to the ingestion of postoperative dietary supplements. Numerous osteoinductive organic compounds can also be considered for the organic doping of the PCL-bioglass hybrid, in particular in the family of the polyphenols (oleuropein, hesperidin, naringin, resveratrol).

Example 8: In vivo evaluation of the implants obtained in examples 1 and 2.

Respect for Ethics and the Animals

All experiments were performed following a protocol approved by the Animal Welfare Committee of the University of Paris Descartes (project approval 17-093, APAFIS No 2018031514511875). The animals were treated according to the ethical conditions developed by the European Union Council Directives (agreement on animal breeding C92-049-01). Every effort was made to minimize their pain or discomfort. The mice C57b16 were purchased from Janvier Labs (Le Genest Saint Isle, France). They were housed at 22±2° C. with a 12-hour day/night cycle and an ad libitum access to food and water in the building of the Department of Orofacial Pathology, Imaging and Biotherapy of the Descartes University, Montrouge, France.

Surgical Implantation, Sampling and Experimental Procedure

The mice C57b16 (12 weeks old, approximately 30 g) were anesthetized by intraperitoneal injection of ketamine (80 mg/kg) and xylazine (10 mg/kg), both from Centravet Alfort, Maisons Alfort, France.

The scalp skin was incised and the periosteum was removed to visualize the skull. A critical size defect of 3.5 mm diameter of the skull cap was created on each side of the parietal bone using a Tissue® punch (from Praxis l′Instrumentiste, France) linked to a slow speed handpiece operating at 1500 rpm, under sterile saline solution irrigation.

Special care was taken for the preservation of the sagittal suture, and a minimal invasion of the dura mater.

After gently removing the bone plug, the defects were filled with the implant made of the hybrid material doped with the fisetin (BG-PCL-Fis) obtained in the example 2 or the undoped implant of the example 1 (BG-PCL).

The dimensions of the cylindrical implants were 3.5 mm in diameter and 1 mm in height (n=8).

For each animal, the same type of material was implanted in both defects.

The defects created in the skulls of six additional mice were left empty as negative controls (“Sham” surgery) to control the criticality of the defect.

The closure of the incisions is completed with an absorbable suture (Vicryl Rapid® 4.0, Ethicon, Johnson & Johnson). An immediate postoperative care was applied by analgesia with buprenorphine (0.02 mg/kg body weight). After surgery, the animals were housed individually under constant observation.

No lethality was observed during the surgery and the postoperative period.

The healing progressed without any signs of infection, exposure of material or other complications.

Body weights were monitored regularly to ensure an adequate nutrition before and after the surgery.

On days 0, 30, 60, and 90 post-surgery, the skull of the animals were visualized by micro X-ray tomography (micro-CT) as described below.

All the animals were euthanized on day 90 and their skull caps were excised.

The samples were fixed in 70% v/v ethanol (24 h at 4° C.), then dehydrated in ethanol solutions comprising increasing %, by volume, of ethanol to remove all traces of water, and embedded at −20° C. in a methyl methacrylate resin (Merck) without decalcification.

The bone samples of the resin coated skull cap were cut (5 mm thick) using a Jung Polycut E® microtome (Leica) with hard tissue blades (Leica).

After immersion in a drop of 50% v/v ethanol, the sections were stretched to a wrinkle-free state on gelatin-coated glass slides (Menzel-Gläser), coated with a polyethylene foil, and pressed tightly onto the glass slides and left to dry overnight at room temperature.

A deplastification was carried out in 2-methoxyethyl acetate (Carlo Erba) three times for 20 minutes.

A rehydration of the sections was performed with ethanol solution of decreasing concentrations for the following procedures.

### X-Ray Micro-Tomography

The mice were anesthetized (isoflurane, 3-4% induction under 0.8 to 1.5 L/min airflow; 1.5 to 2% under 400 to 800 mL/min after) at the baseline, day 30, day 60, and day 90, and were analyzed using an X-ray computed tomography device (Quantum FX Caliper®, Life Sciences, Perkin Elmer, Waltham, Mass.) from the PIV Platform, EA2496, Montrouge, France.

The X-ray source was set at 90 kV and 160 μA.

The three-dimensional images were acquired with an isotropic voxel size of 20 μm.

An internal density phantom, calibrated in mg of hydroxyapatite, was used to scale the bone density.

The high-resolution 3D raw data were obtained by rotating both the x-ray source and the flat panel detector 360° around the specimen (scan time of 3 min).

The three-dimensional renderings were then extracted from the Dico data frames using the OsiriX® imaging software (b5.7.1, distributed under LGPL license, Dr. A. Rosset, Geneva, Switzerland).

The quantification of the regenerated bone within each defect was performed using the CTscan Analyzer® software (Skyscan, version 1.13.5.1, Kontic, Belgium).

A global volume of interest (VOI) was plotted by interpolating the 2D region of interest over consecutive sections to isolate the initial defect area.

The resulting interpolated VOI comprised only the remodeled bone defect area.

A global threshold was interactively determined for the bone selection and to remove the background noise.

The volumetric fractions of “new tissue” were expressed relative to the total volume of the initial defect;

Statistics

The results in each group were expressed as the mean value ±the standard deviation. A Fischer test is used to statistically discriminate the biological groups.

Results

FIG. 4 shows in graphical form the cell viability results of the primary rat osteoblasts in the presence of a control (100%), of BG-PCL, and of BG-PCL-Fis.

The measurement of the viability of the cell activity is based on the XTT activity after 7 days of culture.

In this figure, the mitochondrial activity is expressed as a percentage of the control condition (CTRL).

As can be seen in FIG. 4, no significant difference in the cell viability is demonstrated between the control and the implant according to the invention or according to the prior art.

This lack of significant difference demonstrates the lack of adverse effects of the BG-PCL and BG-PCL-Fis dissolution products on the cell growth and proliferation.

The behaviour of the primary osteoblasts on slices of human cortical bones, of BG-PCL disks from the example 1, and of BG-PCL-Fis disks from the example 2, was studied by scanning electron microscopy.

The human cortical bone slices were used as controls.

FIG. 5 shows the scanning electron microscopy images of RPO grown on:
Top photographs: the human cortical bone slices at 2 different magnifications,
Middle photographs: the BG-PCL disks,
Lower photographs: the BG-PCL-Fis disks.

As can be seen in FIG. 5, in all cases, the cells covered the surface of the materials, showed star shapes and connected filopodia, demonstrating thus a proper cell adhesion.

To be noted, the cells seem to spread more efficiently on the BG-PCL surface and even more so on the BG-PCL-Fis surface, compared to the human cortical bone, demonstrating the creation of an environment that favours the cell adhesion of the doped hybrid material of the invention.

More importantly, the computed tomography images reproduced in FIG. 6 show that after 30 days:
the empty defects (control left empty) show a low bone regeneration of about 10% (insufficient spontaneous regeneration, defining the critical character of the defect),
the defects filled with BG-PCL have a significantly higher bone volume of approximately 30%, and
the defects filled with BG-PCL-Fis achieve a bone regeneration of 55%.

At the end of the 90-day trial, the bone volume reached approximately 15% in the controls while more than 30% of the bone defects are repaired using BG-PCL implants.

Remarkably, when using the implant doped with the fisetin BG-PCL-Fis of the example 2, the new bone formation extended to the entire defect and covered more than 55% of the initial defect.

These results are shown as curves in FIG. 7.

1. An implant material for filling bone defects, bone regeneration and bone tissue engineering,
characterized in that
it comprises a hybrid material comprising:
a bioactive glass M made from SiO₂ and CaO, optionally containing P₂O₅ and/or optionally doped with strontium, and
a biodegradable polymer P is selected from:
the bioresorbable polysaccharides, preferably selected from dextran, hyaluronic acid, agar, chitosan, alginic acid, sodium or potassium alginate, galactomannan, carrageenan, pectin,
the bioresorbable polyesters, preferably polyvinyl alcohol or polylactic acid, and
the biodegradable synthetic polymers, preferably a polyethylene glycol, poly(caprolactone)
and in that this hybrid material is doped with an osteoinductive nutrient N selected from vitamin D2 (ergocalciferol), vitamin D3 (cholicalciferol), vitamin K1, vitamin K2, omega-3 fatty acids, punicic acid, α-lipoic acid, anthocyanins, flavonols, procyanidins, tyrosol, oleuropein, naringenin, punicalagin, ellagic acid and phycocyanin.

2. The implant material for filling in bone defects, bone regeneration and bone tissue engineering according to claim 1, characterized in that the hybrid material doped with an osteoinductive nutrient comprises 30% by weight of bioactive glass M made from SiO₂ and CaO, relative to the total weight (bioactive glass M+biodegradable polymer P+osteoinductive nutrient N), 69% by weight of poly(caprolactone), relative to the total weight (bioactive glass M+biodegradable polymer P+osteoinductive nutrient N), and 1% by weight of fisetin and/or hydroxytyrosol, relative to the total weight (bioactive glass M+biodegradable polymer P+osteoinductive nutrient N).

3. The implant material for filling in bone defects, bone regeneration and bone tissue engineering according to claim 1, characterized in that the hybrid material doped with an osteoinductive nutrient comprises 40% by weight of bioactive glass M made from SiO₂ and CaO, relative to the total weight (bioactive glass M+biodegradable polymer P+osteoinductive nutrient N), 59% by weight of poly(caprolactone), relative to the total weight (bioactive glass M+biodegradable polymer P+osteoinductive nutrient N), and 1% by weight of fisetin and/or hydroxytyrosol, relative to the total weight (bioactive glass M+biodegradable polymer P+osteoinductive nutrient N).

4. An implant made of a hybrid material for filling in bone defects, bone regeneration and bone tissue engineering, characterized in that it comprises a material according to claim 1.

5. A method for manufacturing an implant made of a hybrid material for filling in bone defects, bone regeneration and bone tissue engineering, characterized in that it comprises the following steps:
a) selecting a bioactive glass M made from SiO₂ and CaO, optionally containing P₂O₅ and/or optionally doped with strontium,
b) selecting a biodegradable polymer P which is soluble in at least one solvent S1 and insoluble in at least one solvent S, selected from:
the bioresorbable polysaccharides, preferably selected from dextran, hyaluronic acid, agar, chitosan, alginic acid, sodium or potassium alginate, galactomannan, carrageenan, pectin,
the bioresorbable polyesters, preferably polyvinyl alcohol or polylactic acid, and
the biodegradable synthetic polymers, preferably a polyethylene glycol, or poly(caprolactone)
c) selecting microspheres of a pore-forming agent A having diameters and sizes corresponding to the desired diameters and sizes of the pores in the material constituting the implant to be manufactured, this pore-forming agent A being:
made of a polymer insoluble in the at least one solvent S1 and soluble in the at least one solvent S,
d) selecting at least one osteoinductive nutrient N:
soluble in at least one solvent S2 identical or different from the solvent S1 but miscible with the solvent S1, that degrades neither the biodegradable polymer P nor the bioactive glass M, and in which the microspheres of pore-forming agent A are not soluble, and
insoluble in the solvent S,
selected from vitamin D2 (ergocalciferol), vitamin D3 (cholicalciferol) and vitamin K1, vitamin K2, omega-3 fatty acids, punicic acid, α-lipoic acid, anthocyanins, flavonols, procyanidins, tyrosol, oleuropein, naringenin, punicalagin, ellagic acid and phycocyanin,
e) introducing microspheres of the pore-forming agent A into a mould having the desired shape and size for the implant, these microspheres forming a compact stack corresponding to the shape and the size of the pores to be obtained in the implant material, and representing at least 60% by volume, preferably at least 70% by volume relative to the total volume of the pore-form ing agent A-biodegradable polymer P-alkoxide precursors of the bioactive glass M-osteoinductive nutrient N mixture,
f) introducing the osteoinductive nutrient N, in solution in the solvent S2, into the biodegradable polymer P in solution in the solvent S1, and mixing,
g) introducing the mixture obtained in the step f) into the alkoxide precursors of the bioactive glass M,
h) introducing the mixture obtained in the step g) into the mould,
i) solidifying the mixture contained in the mould after the step h),
j) demoulding the mixture obtained in the step i),
k) removing the microspheres of pore-forming agent A by washing with the solvent S.

6. The method according to claim 5,
characterized in that the material of the pore-forming agent A is selected from the biodegradable polymers insoluble in the at least one solvent S1 and the at least one solvent S2 and soluble in the at least one solvent S, preferably selected from C₁ to C₄ alkyl polymethacrylates, preferably methyl polymethacrylate or butyl polymethacrylate, polyurethane, polyglycolic acid, the various forms of polylactic acid, the lactic-coglycolic acid copolymers, poly(caprolactone), polypropylene fumarate, paraffin and naphthalene, or acrylonitrile butadiene styrene (ABS),
the material of the pore-forming agent A being different from the biodegradable polymer P.

7. The method according to claim 5, characterized in that the weight ratio of biodegradable polymer P to bioactive glass M is between 20/80 and 80/20, inclusive, and that the osteoinductive nutrient N is present in an amount between 0.1 and 10%, preferably between 0.1% and 5%, more preferably of 1%, by weight relative to the total weight of the material obtained in the step k).

8. The method according to claim 5 characterized in that:
the bioactive glass M is a glass made from SiO₂ and CaO,
the biodegradable polymer P is the poly(caprolactone),
the osteoinductive nutrient N is the fisetin and/or the hydroxytyrosol,
the material of the microspheres of pore-forming agent A is the paraffin,
the solvent S is the cyclohexane,
the solvent S1 is identical to the solvent S2 and is the tetrahydrofuran (THF).
